(19) United States
(12) Patent Application Publication
(10) Pub. No.: US 2025/0325004 A1
(43) Pub. Date: Oct. 23, 2025

(54) Transfer of Substances with Dietary and Medicinal Properties from Biomass after Mushroom Cultivation to Insect Bodies

(71) Applicant: KONRAD WLODARCZYK, MASLOMIACA (PL)

(72) Inventor: KONRAD WLODARCZYK, MASLOMIACA (PL)

(21) Appl. No.: 18/637,458

(22) Filed: Apr. 17, 2024

(51) Int. Cl. A23K 10/30; A01K 29/00; A01K 67/033; A23K 50/90

(52) U.S. Cl. CPC A23K 10/30; A01K 29/00; A01K 67/30; A23K 50/90

(57) ABSTRACT

Insects with elevated levels of protein, chitin, elements, amino acids, vitamins, and/or sterols are disclosed. The insects have elevated levels due to a diet that includes mushroom biomass. The mushroom biomass includes any one or more of the mushroom fruitbody, the stem, and the mycelium. The enriched insects with biomolecules from mushroom biomass can be used as they are or as supplements to increase the levels of biomolecules in humans or other animals that may ingest the insects or a modified form of the insect.

### CROSS-REFERENCE TO RELATED APPLICATIONS

The present application claims priority under 35 USC 119 (e) to U.S. Provisional Application No. 63/460,622 filed Apr. 20, 2023, the entire contents of which is incorporated by reference.

### FIELD OF THE INVENTION

The present invention relates to methods and products developed from the technology of transferring substances from the biomass of mushrooms after their cultivation to the body of insects. Insects of the order Coleoptera, Diptera and Orthoptera in their larval form are particularly suitable for this transfer, with transfer best attained in the rearing of the larval stage of the insects. The transfer of these substances should be attainable on an industrial scale and the amounts of the substances can significantly increase the content of these components in insect bodies relative to control insects, significantly enriching the final products obtained from it.

### BACKGROUND OF THE INVENTION

Rearing and breeding of insects on an industrial scale is one of the most dynamically developing new branches of agricultural production and the biotechnology industry. The high content of protein, vitamins, elements and all essential amino acids, their extraordinary ability to convert low-energy feed from agricultural and food industry by-products in a low-emission process into high-quality protein and fat, combined with a short fattening period, places insects at the forefront of the reconstructed food chain.

World production of mushrooms will reach 24 million tons per year in 2027. (Source: https://www.marketdataforecast.com/market-reports/button-mushrooms-market, (See https://www.fortunebusinessinsights.com/industry-reports/mushroom-market-100197)

In Europe and North America, the main cultivated mushroom is the button mushroom (Agaricus bisporus). For every kilogram of ready-to-eat mushrooms, over 2 kg of growing medium is usually needed. However, after cultivation, the SMS (spent mushroom substrate) or SMC (spent mushroom compost) which is completely overgrown with mycelium and contains most of the mushroom's residual nutrients. To date, no satisfactory mechanical or chemical method has been found to separate the mycelium from the substrate and use the wealth of ingredients contained in the mycelium. Mycelium is often composted and used as a soil, which not only wastes the specific substances contained in the mycelium, but it also increases the carbon footprint of mushroom cultivation.

The case is similar with the cultivation of arboreal/woodland mushrooms. They are cultivated by inoculating with mycelium bales of various sizes, consisting primarily of shavings of deciduous trees, cereals such as wheat, corn, soy hull, sorghum, and their milling products such as bran and even nut shells, hemp straw, coconut, vermiculite and/or cardboard. The following species are mainly cultivated in this way: oyster mushrooms (Pleurotus ostreatus, Pleurotus eryngii), shiitake (Lentinula edodes), pioppino (Agrocybe aegerita), Lion's Mane (Hericium erinaceus), Buna shimeji (Hypsizygus tessulatus), and enoki (Flammulina velutipes).

In this technology, used bales are a waste product entirely overgrown with mycelium that must be discarded. Because the harvesting of mushrooms is usually done in a single break/phase, the amount of side stream is as large (or sometimes larger) relative to the amount found when harvesting button mushrooms. Given that in some mushrooms dietary and medicinal active compounds are only found in the mycelium, e.g. erinacines in Lion's Mane (Hericium sp.), any recovery that can be attained by a transfer to insects would be advantageous particularly because it is efficient and low-cost.

For each kilogram of mushroom ready for sale, 15-25% of material from the harvesting and processing waste is the stems. This biomass is characterized by a high content of nutrients and, like the substrate, is not managed or conserved in any way.

Another waste product in traditional mushroom production is water, which is procured after rinsing mushrooms as well as attained as a byproduct of the water used to blanch mushrooms. This waste water tends to be extremely rich in protein and the fluids are also very nutrient rich.

Another source of liquid biomass is attained after fungal cultivation, wherein the liquid/media from the culture of in vitro mycelial crops and micro-fungus of the genus Fusarium can be collected from mycoprotein bioreactors. This type of fungal cultivation is gaining popularity and is considered by much of the scientific community and industry to have a more sustainable future for the cultivation of edible, medicinal mushrooms or for Fusarium venenatu. The cultivation of Fusarium venenatum has one of its strains used commercially for the production of the single cell protein mycoprotein Quorn.

Due to the feeding characteristics of fungi, which are adulterous organisms, which first digest and then eat by secreting digestive enzymes into their external environment, the fluids after in vitro cultivation contain an extraordinary wealth of nutrients, making them ideally suited for medicinal purposes.

In mycoprotein bioreactors, on the other hand, the broth/substrate after mycoprotein production using the Fusarium venenatum species (in either Quorn or Airlift fermenters) is centrifuged to produce a “mycelium paste” containing approximately 75% water. The resulting product is called mycoprotein, which is rapidly cooled and in some embodiments, ready for use in the production of food products.

In the biomass that is produced, the amount of RNA is reduced so that the product meets the requisite health standards. Reducing the RNA content is achieved by subjecting the biomass to heat shock at a temperature of around 64-65° C. and keeping the biomass in a separate reactor with a stirrer for around 20-30 minutes. At this temperature, the RNA is degraded into nucleotide or nucleic acid monomers and diffuses out of the cell. Unfortunately, at this temperature, other biomass components also decompose, which leads to the loss of approximately 35-38% of the mass of the potentially useful product. After RNA content is reduced, the liquid from the reactor together with the biomass is heated to 90° C. The biomass is then centrifuged and cooled.

The effect of the RNA reduction step is leakage through the cell membrane, resulting in the loss of up to or more than 30% of the biomass to the filtrate (i.e., the liquid drained from the centrifuge), or sidestream, in the form of liquid biomass.

The composition of the filtrate is shown in tables A and B below:

TABLE A
Average amino acid composition for a typical supernatant/side
stream (grams per 100 grams dry matter)

| | | | | | |
|---|---|---|---|---|---|
| Aspartic Acid | 1.6 | Threonine | 0.8 | Methionine | 0.3 |
| Serine | 0.8 | Alanine | 1.9 | Lysine | 1.2 |
| Glutamic Acid | 4.7 | Proline | 0.8 | Isoleucine | 0.6 |
| Glycine | 1.3 | Cysteine | 0.3 | Leucine | 1.0 |
| Histidine | 0.5 | Tyrosine | 0.4 | Phenylalanine | 0.6 |
| Arginine | 1.4 | Valine | 0.8 | | |

TABLE B
Average typical values of the chemical composition
of the supernatant/ side stream.

| | Concentration (g/L) | Dry matter (%) |
|---|---|---|
| Carbohydrates (total) | 4.8 | 37 |
| Nucleic Acids (5′ GMP) | 1.6 | 12 (2-3%) |
| Proteins (amino Nx6.25) | 2.2 | 17 |
| Inorganic Salts | 3.6 | 28 |
| other | 0.8 | 6 |
| total | 13 | |

The filtrate/side stream will typically have a total solids content of about 1.3 g/100 mL, and is therefore a dilute biomass containing the biomolecules of interest.

Dewatering/extraction of the fermenter “waste”, known as the filtrate or side stream, produces a powdered 5′-nucleotide-rich ingredient that can be used as a yeast-free flavor enhancer in foods with a proven umami effects/flavor.

Research is also focusing on ways to partially divert the leachate (water that has percolated through a solid) for use in fermentation, thereby providing a means of reducing effluent loading and water consumption, and further increasing the sustainability of the process.

However, both processes tend to be extremely expensive and energy-intensive, and due to the high processing temperatures that are required, they lose most of their medicinal ingredients due to degradation.

In contrast, the leachate generally can be used without dewatering/extraction as a liquid ingredient in insect feeds to transfer components with dietary and medicinal properties to the insect bodies. This transfer is analogous to a transfer of solid biomass.

The general use of biomass produced by various species of mushrooms and feeding it to insects as a means of waste disposal are known. However, prior to the instant invention, to the inventor's knowledge, the use of biomass resulting from the cultivation and processing of mushrooms in insect feed to improve the quality of the final product in the form of insect meat and/or crude protein, fat and chitin, and the biotechnological transfer of substances contained in the mycelium to insect protein and intensification of the content of these substances in insect body is heretofore unknown. The present invention takes advantage of the high content of biomolecules in mushrooms and its effective ingestion by insect larvae in order to enrich the final products from them.

### BRIEF SUMMARY OF THE INVENTION

The present invention relates to transferring substances from the biomass of mushrooms after their cultivation (in particular, the common mushroom Agaricus bisporus) to the bodies of Coleoptera, Diptera and Orthoptera insects. The present invention also relates to the rearing and breeding of these insects on an industrial scale in order to significantly increase the content of these components in insect bodies.

### BRIEF DESCRIPTION OF THE SEVERAL VIEWS OF THE DRAWING

FIG. 1 shows a generalized scheme of insect larvae growth using mushroom biomass.

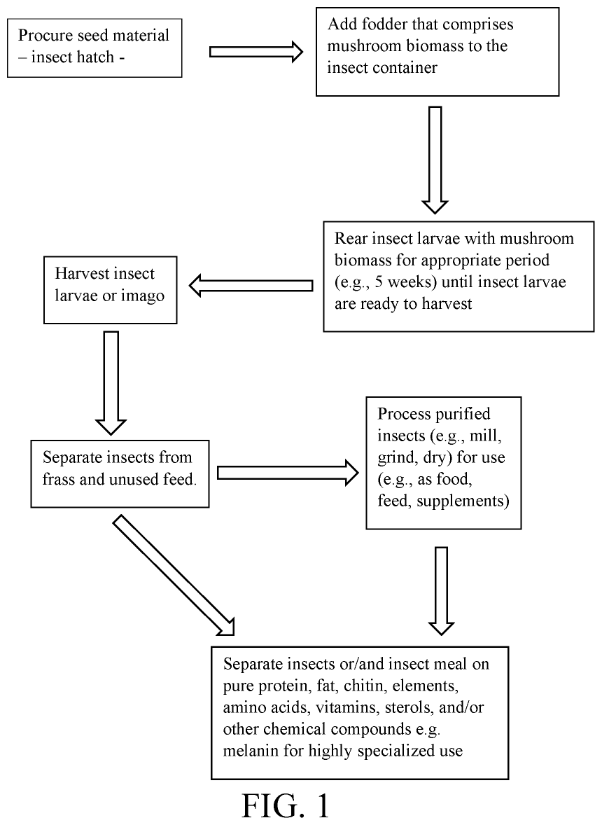

### DETAILED DESCRIPTION OF THE INVENTION

In an embodiment, the present invention relates to increasing the bio-elements present in insect body. Insects in the larval form obtained in the process of rearing and breeding according to the invention comprise and are characterized by an increased content of bio-elements, especially macro-elements (K, Ca) and microelements (Cu, Fe, Zn) when fed fodder supplemented with cultivated mushroom relative to insects that are fed fodder that do not contain cultivated mushroom. These substances are vitally important for all higher order organisms. The insects in the larval form also have a higher content of exogenous amino acids such as phenylalanine and tryptophan. In an embodiment, phenylalanine and L-tryptophan are present at higher amounts, which make them a particularly valuable material that can be used as a dietary supplement in human food, pet food and farm animal diets.

The production technology of different species of insects is often fundamentally different and the use of one technology for production is not necessarily transferable to other technologies. In the Coleoptera insect order, the insects undergo “dry breeding” in which the breeding and rearing substrate and at the same time the dry base feed of the larval forms are cereal grains, flour obtained from milling these cereals, bran and all milling by-products. In “dry farming”, water is supplied to insects most often in the form of endogenous water included in the plant component. The water is supplied endogenously in all food stuffs including but not limited to its presence in all vegetables, fruits, their parts, other by-products of agri-food processing, including all products of plant origin, in their fragmented form, in pulp, gel or jellies.

Starting from the beginning of the life cycle, a dry base mix (usually in the form of cereal bran) is the substrate in which the adult insects lay their eggs. The eggs are incubated in the dry base mix, and it is this mix in which the larvae are then fattened until processing. As the dry mix is both the base feed and the substrate that increases the living space of insects, the feed is successively eaten throughout the fattening period, with insect frass gradually replacing the proportion of dry mix feed. In the final stage of fattening, there are only ready-to-process larvae and insect frass in the breeding containers.

The “dry” method of breeding and cultivation is also used in the case of insects from the Orthoptera order. The only exception here is frass, which does not constitute a living space for larval forms and should be systematically removed. However, the remaining technology of feeding with dry base feed and wet fruits/vegetables in their fragmented form, in pulp, gel or jellies or just water remains unchanged.

In turn, in the “wet” technology most often used in the fattening of insects from the order of Diptera, the larvae stay in a wet environment consisting most often of vegetables, fruits, meat products or even manure and their parts, which, as in the case of dry technology, constitute both their living space and fodder. These food stuffs are successively converted into a by-product of metabolism, i.e., frass.

The substrate that is present after the cultivation of the two-spore mushrooms (SMS/SMC) as well as the processing waste in the form of stems can be used at every stage of production of the various insect species. The mushrooms' processing waste and SMS are used in both dry and wet technologies.

In order to feed insects, the substrate together with the casing soil after the final harvest of mushrooms can be subjected to thermal steam disinfection, usually the substrate is heated to 80° C. for 6-8 hours. At any time in the disinfection process, the casing can be mechanically separated from the substrate or it can be left completely unseparated. If the casing soil is not separated, the addition of peat helps maintain the appropriate moisture of the insect's rearing substrate and enriches the frass. In the case of separation, the casing can be recycled and reused. The substrate with or without casing is mechanically crushed into fractions wherein the particle size is between about 0.01 mm to 20 mm. The crushing can be accomplished by using any device. For example, the crushing device may be one or more of an industrial grinder, grinding machine or a blender. The crushed material can then be tested and proved by adding the appropriate amount of water for the species (usually in the ratio of 1:1-5) to obtain a homogeneous mass.

For process reasons, the sequences can also be advantageously reversed by first hydrating the material and then grinding it to the desired fraction size.

The material can be supplemented with any liquid, e.g. water (including industrial water), juice or in a variation, the liquid after mushroom rinsing and/or blanching and/or mycelial, and/or alternatively, mycoprotein liquids.

The material suitable for use is the entire waste after harvesting the last cultivation phase in the form of SMS, casing soil, and stems grown in raw form or after the disinfection process. After grinding, such material usually does not require watering due to the high endogenous water content of the stems themselves. This material is suitable for feeding in the form of fresh pulp in both dry and wet technologies. The raw material prepared in this way can also be processed into a gel or jelly using techniques that inhibit the process of pulp decomposition. As a result of experiments that were conducted, it was found that extremely fast decomposition of the mycelium, although it does not have a negative effect on the fattening of the insects themselves, leads to a reduction in the content of some ingredients, e.g. proteinogenic amino acids. This reduction prevents their transfer. A slower decomposition process allows ingredient amounts to be higher.

In any event, the prepared feed ingredient should be applied in accordance with the needs and the production technology used.

Processing waste derived from the stems of mushrooms is used in a similar manner to the products derived from mycelium. Stems are obtained in the mushroom harvesting process. As is the case with fruit and vegetable pulp, the stem material should be shredded in any of a plurality of ways to generate a fraction size that is between about 0.01 mm and 20 mm. Alternatively, fractionation may occur to achieve sizes that are between about 0.1 to 20 mm, or alternatively between about 1 mm to 10 mm or between about 2 mm to about 5 mm. Due to its high-water content, in an embodiment, one can thicken this material either with food and/or feed thickeners or other feed ingredients, e.g. corn flour, whole ground corn kernels or by adding a feed supplement in the form of barley brewer's grain (dry or wet). For thickening, one can also use another type of fungi in the form of feed yeast or yeast slurry, which is a byproduct from brewing beer, wine, or liquors.

The material obtained usually undergoes very rapid decomposition, even at low temperatures, such as at refrigeration temperatures. This rapid decomposition to some extent deprives the processed mushrooms of the components that are desired to be transferred. In an embodiment, to slow down the decomposition process, stems can be subjected to shock cooling at 2-4° C. immediately after harvesting, using any of a plurality of techniques including but not limited to temperature reduction techniques as well as vacuum cooling through pressure reduction. The raw material obtained in its pulp form should be stored under cold conditions and fed to insects as soon as possible after processing. In a variation, one way to extend its shelf life is to process the raw material into a gel or jelly using preservative additives.

As both the SMS and stems are a waste/by-product of mushroom cultivation, they can usually be mixed together. The raw material can also be used. Depending on the relative amounts of stems, the resulting mix will have different degrees of hydration. In an embodiment, after grinding and fractionating the material to a size of between about 0.10 mm to 20 mm, water can be added depending on the desired hydration level. In an embodiment, the amount of water that is added is to give the pulp the desired and appropriate physical and chemical parameters.

The method of rearing and/or breeding of insects according to the invention comprises and is characterized by the fact that the material according to the invention is used at various developmental stages of insects. The material of the present invention can also be used in different technologies, it can be used as a feed, as a feed additive/supplement, or as a substrate. It can be combined with any other component or material. In one embodiment, it is used for transferring ingredients from the biomass of mushrooms after their cultivation and processing into insects. The insects will effectively process the biomass from mushrooms, thereby making the components that are initially present in mushrooms that have dietary and medicinal benefits available to humans and other higher order animals.

The following section illuminates some of the advantages of the present invention.

### Advantages of the Invention

In one embodiment, the present invention provides increased amounts of useful molecules in insect larvae and/or one or more final products that are enriched. In a variation, one benefit of the present invention is the increased amounts of individual ingredients, such as magnesium, calcium, tryptophan, etc. Because these ingredients have recommended daily allowances and are important for human health, any vehicle that makes these molecules more readily available in higher content is useful. For example, sometimes the various molecules can be used for their healing effect in humans and other animals.

The final products enriched in this way potentially contain enhanced antioxidant, anti-aging, anti-inflammatory, regenerating, vitalizing, neuroprotective and antidepressant properties, enabling their wide use in nutraceuticals, supplements for athletes and convalescents, pharmaceuticals as well as potentially being used in advanced medicines and cosmetology.

The healthy and nutritional content of ingredients found in enriched insect protein also enables its widespread use to generate the highest quality products for farm-animal feed and aquaculture. They provide better ingredients for highly specialized pet foods as well as veterinary nutrients and supplements.

Moreover, the increased content of chitin and melanin enables innovative applications in heavy industry, such as in the biodegradable electronics or energy storage fields.

### Examples of Implementation of the Invention

I. In an embodiment, an experiment was conducted with the aim of demonstrating the differences in the content of selected bio-elements in the body of Alphitobius diaperinus larval forms between individuals fed with traditional feed mixtures and those fed traditional feed mixtures that were supplemented with a mushroom biomass additive that was attained after cultivation and processing of A. bisporus mushrooms.

The insects used in the various experiments came from breeding derived from insects that were present in the inventor's inventory. The experiments were carried out throughout the rearing period of the larval forms, i.e., at time periods after the hatching of eggs. The larvae were allowed to grow using the feed disclosed herein for a period of 5 weeks. The experiments were carried out using 8 repetitions, each time on 100 breeding containers for each group, and the various experiments showed no noticeable difference in their BWG (Body Weight Gain) values-their weight gain, their FI (Food Intake)—the amount of food consumed, their FCR (Food Conversion Ratio) or their length of rearing. Thus, the differences that are seen herein are attributed solely to the differences in the food stuff that each larval group had available. The results reported herein have been averaged.

Insects were reared in polypropylene containers with a bottom surface area of 0.24 m² at a temperature of 30° C. and 60% humidity throughout the fattening period, in the dark. The same starting amounts of beetle eggs were used each time. The 5-day incubation period was followed by a 5-week fattening period. The feed was fed at 56 hour intervals depending on demand. The mass of insects was determined after separating them from frass and the food remains were weighed at the end of the cycle. No dead insects were found in any of the samples. The following feed mixtures were used:

### A. Feed Mixture Composed of:

−35% by mass wheat bran, 60% by mass carrot pulp, and 5% by mass feed supplement in the form of barley brewer's spent grains.

### B. Compound Feed Composed of:

−35% by mass wheat bran, 60% by mass pulp from mushroom biomass after last cut of cultivation (contains SMS residue and mushroom stems), and 5% by mass feed supplement in the form of barley brewer's spent grain.

The pulp from the substrate and stems of mushrooms, prepared as described, with a degree of hydration in the range of 70-90%, was applied immediately after preparation at 56-hour intervals. At the end of the fattening period, the larvae were separated from the frass and the rest of the unfinished feed. The larvae were dried and then ground. Samples were obtained from the material prepared in this way and then analyzed.

The biomass obtained was homogenized in an agate mortar and subjected to the mineralization process. Nanohunter II, a Rigaku (Japan) X-ray fluorescence spectrometer (TXRF) was used to analyze the elemental composition of the samples, using the total reflection of X-ray radiation. The Nanohunter II instrument contains an X-ray source that is a 600 W molybdenum tube and is equipped with a 16-sample loader that ensures automatic operation of the device.

The results obtained verified that transfer of bio-elements to insect larvae from feed occurs in all situations. However, the addition of Agaricus bisporus post-production residue was found to give higher concentrations of almost all of the desired elements (see Table 1). Table 1 shows the content of selected elements in insect larvae fed with traditional fodder for the full fattening period (Insects A) and fed with fodder with the addition of biomass after the cultivation of mushrooms depending on the stage of development. The group that is shown as Insects 1 was grown for ⅔'s of the fattening period, whereas the group that is shown as Insects 4 was grown for the full fattening period. Results are shown as mg of the respective element/kg dry weight.

TABLE 1

| Element | K | Ca | Cr | Mn | Fe | Cu | Zn | Se | Rb | Sr |
|---|---|---|---|---|---|---|---|---|---|---|
| INSECTS | 8006 ± 186.6 | 401.7 ± 9.3 | 1.6 ± 0.4 | 6.4 ± 0.2 | 46.9 ± 1.2 | 18.0 ± 0.6 | 174.7 ± 0.5 | 173.5 ± 0 | 0.9 ± 0.1 | 2.3 ± 0.2 |
| A | | | | | | | | | | |
| INSECTS | 8379.2 ± 218.4 | 343.5 ± 64.2 | 1.4 ± 0.3 | 6.6 ± 0.2 | 56.7 ± 0.6 | 18.6 ± 1.1 | 193.0 ± 0.8 | 172.2 ± 0 | 0.6 ± 0.04 | 1.1 ± 0.04 |
| 1 | | | | | | | | | | |
| INSECTS | 8791.1 ± 255.3 | 508.1 ± 8.1 | 2.6 ± 0.1 | 6.5 ± 0.1 | 87.4 ± 1.1 | 28.3 ± 0.1 | 203.3 ± 1.2 | 135.1 ± 0 | 1.5 ± 0.01 | 0.8 ± 0.01 |
| 4 | | | | | | | | | | |

N = 6, Se internal standard

A review of the table shows that almost all of the elements increased by feeding the insects mushroom biomass (after cultivation and processing described herein). The analysis showed the following percentage increase in the content of elements:
Potassium-content increase by 6.7%
Calcium-content increase by 69%
Manganese-content increase by 12%
Iron-content increase by 37.6%
Copper-content increase by 5.99%
Zinc-content increase by 10.4%
Rubidium-content increase by 66.6%

As can be seen from the results of Table 1, one will note that the transfer of macro-elements (K, Ca) and microelements (Cu, Fe, Zn) is elevated in the groups that are fed the fodder that is supplemented by the mushroom biomass. These elements are known to be vitally important to the human body and to the health of humans. Accordingly, the use of the insect larvae with elevated levels of the respective elements should make them particularly valuable in a diet. Of note, the tested material has a Zn content that is sufficiently high that it could easily be used as a dietary supplement to easily meet the daily human demand for this element. Similarly, insect larvae with elevated levels should serve as a good source of Fe, which also can be used as a supplement to the human diet. Both groups comprising the fodder supplemented by the mushroom biomass showed superior results in these elements relative to the control group. Higher levels of various most of the elements were seen in the larvae that had their fodder supplemented by mushroom biomass. Subsequent testing was done to ascertain if the insect larvae also saw comparable results with necessary dietary organic molecules.

II. The aim of the next experiment was to ascertain if there were differences in the content of selected organic compounds in the bodies of Alphitobius diaperinus larvae between individuals fed with traditional feed mixtures and with the supplement of the biomass derived from A. bisporus after its cultivation and the harvesting process.

The insects used in the experiments came from the inventor's breeding inventory. The experiments were carried out each time throughout the fattening period of larval forms, i.e. from the time period starting with the hatching from eggs to the time period when the larval forms are mature (and are ready for processing) after a period of 5 weeks. The experiments were carried out in 8 repetitions, each time on 100 breeding containers for each group, and the respective groups showed no differences in the BWG (Body Weight Gain) values-weight gain, FI (Food Intake)—amount of food consumed, FCR (Food Conversion Ratio) or length of fattening. Accordingly, the differences seen in the relative amounts of the various biomolecules can be attributed to the different diets. The results have been averaged.

Insects were reared in polypropylene containers with a bottom surface area of 0.24 m² at a temperature of 30° C. and 60% humidity throughout the fattening period, in the dark. The same starting amounts of beetle eggs were used for each group. The 5-day incubation period was followed by a 5-week fattening period. The feed was supplied at 56 hour intervals depending on demand. The mass of insects was determined after separating them from their frass and the food remains and the larvae were weighed at the end of the cycle. No dead insects were found in any of the samples. As above, the following feed mixtures were used:

### Feed Mixture Composed of:

A. −35% by mass wheat bran, 60% by mass carrot pulp, and 5% by mass feed supplement in the form of barley brewer's spent grains.
B. Compound feed composed of:
−35% by mass wheat bran, 60% by mass pulp from mushroom biomass after last cut of cultivation (contains SMS residue and mushroom stems), and 5% by mass feed supplement in the form of barley brewer's spent grain.

The pulp from the substrate and stems of mushrooms was prepared as described, with a degree of hydration in the range of 70-90%, with the feed being applied immediately after preparation at 56-hour intervals. At the end of the fattening period, the larvae were separated from the frass and the rest of the unfinished feed. The larvae were dried and then ground. Samples were obtained from the material prepared in this way and then analyzed.

The insects were homogenized in an agate mortar. Samples with appropriate weights were selected experimentally and were extracted for 20 minutes using methanol in an ultrasonic bath with a frequency of 40 kHz (Sonic-2, Polsonic). The extraction was repeated with methanol several times for each sample. The repeated extracts were combined (300 mL) and concentrated. The evaporated extracts were quantitatively dissolved in HPLC methanol and then filtered using membrane filters (Millex, Millipore Corporation, USA). The extracts thus obtained were ready and were used for HPLC analysis.

### L-Tryptophan Analysis

Measurements of L-tryptophan in methanolic extracts were chromatographically performed using an HPLC chromatograph from Hitachi-Merc (Dublin, Ireland). A device with a L-7100 pump, a L-7400 UV-VIS detector and a Purospher® (Dublin, Ireland) column was used to determine indole (e.g., tryptophan) compounds. The temperature of the Purospher® RP-18 column (4 mm×200 mm, 5 μm) was 25° C., and UV detection was recorded at 2=280 nm. The liquid phase used was a mixture of methanol/water/ammonium acetate in volume proportions (15:14:1 v/v/v) and a flow rate of 1 mL/min. Quantitative analyzes of indole compounds were carried out using Beer's law and a calibration curve, assuming a linear relationship between the absorbance and the concentration of the standard. The results were expressed in mg/100 g dm. An isocratic elution program was used for the determination of indole compounds.

The results were calculated from the standard curve of the L-tryptophan standard, the dependence of the area under the peak on the concentration of the compound.

Each of the tested materials was analyzed in 6 independent replicates. The results (see Table 2) of the determinations are presented as mean values with standard deviation (SD).

### Phenylalanine Analysis

Determination of phenylalanine content was carried out by RP-HPLC method using HPLC VWR Hitachi-Merck (Dublin, Ireland) apparatus with L-2200 autosampler, L-2130 pump, RP-18e LiChrospher column (4 mm×250 mm, 5 μm) thermostated at 25° C., L-2350 column detector, L-2455 diode detector operating in the UV wavelength range of 200-400 nm. The mobile phase consisted of solvent A: a mixture of methanol and 0.5% acetic acid by volume (1:4 v/v) and solvent B: methanol. The gradient was: 100:0 for 0-25 min, 70:30 for 35 min, 50:50 for 45 min, 0:100 for 50-55 min, 100:0 for 57-67 min. Comparison of the UV spectra and retention times with a standard (standard) enabled the identification of phenylalanine present in the analytical samples. Quantitative analysis of free phenylalanine was carried out using a calibration curve, assuming a linear relationship between the absorbance and the concentration of the standard (Table 2).

### Ergothioneine Analysis

The high-performance liquid chromatography (RP-HPLC) method (Merck Hitachi, Tokyo, Japan), UV detector L-7400 (Merck Hitachi, Tokyo, Japan), pump L-7100, thermostat L-2350, columns RP-18 4×250 mm (Purospher®, grain size 5 μm) was performed to analyze the ergothioneine amounts.

The analysis was performed using isocratic elution. A mixture of water and methanol in a volume ratio of 99:1 with the addition of 3.0 g of boric acid was used as the mobile phase to bring the solution to pH=5.0. The mobile phase flow rate was set at 0.5 mL/min. 20 μL of sample was dispensed onto the column. Measurements were made for 20 minutes at a wavelength of 257 nm. Quantitative analysis of ergothioneine was carried out using a calibration curve, assuming a linear relationship between the absorbance and the concentration of the standard (see Table 2).

Table 2 shows the content of selected organic compounds in insect larvae fed with traditional fodder for the full fattening period (Insects A) and fed with fodder with the addition of biomass after the cultivation of mushrooms depending on the stage of development. The group listed as Insects 1 were larvae that were grown for ⅔ of the fattening period, and the group named Insects 4 were analyzed after the full fattening period. The various organic compound amounts are given in mg of the biomolecule/100 g of dry weight insect larvae.

TABLE 2

| Relationship | L-Tryptophan | Phenylalanine | Ergothioneine |
|---|---|---|---|
| INSECTS A | 46.8 ± 0.8 | 305.0 ± 6.0 | 1.82 ± 0.12 |
| INSECTS 1 | 100.5 ± 5.6 | 579.3 ± 3.2 | 8.26 ± 0.01 |
| INSECTS 4 | 76.7 ± 1.4 | 502.9 ± 2.8 | 5.15 ± 0.01 |

N = 6

The analysis of the results showed a significant increase in the content of selected amino acids (up to over 450%) between insects fed with biomass after mushroom cultivation and processing and insects fed with traditional feed mixtures. Particularly noteworthy is the fact that the content of amino acids reaches its peak value in ⅔ of the fattening period (before diminishing). Without being bound by theory, it is believed that this result stems from the fact that in the final fattening period, the larval forms use the accumulated amino acids in preparation for transformation into the form of imago.

In an embodiment, the insect larvae, which are fed fodder with mushroom biomass may produce elevated levels of the essential amino acids. The essential amino acids include histidine, isoleucine, leucine, lysine, methionine, phenylalanine, threonine, tryptophan, and valine.

Subsequently, experiments were performed to ascertain the relative amounts of ergosterol that was present in the larval control groups versus those larvae that were fed fodder supplemented with mushroom biomass.

### Ergosterol

Table 3 shows the results of ergosterol content in the larval insects that were fed fodder supplemented with mushrooms relative to the control (i.e., those larval insects fed fodder not supplemented with mushroom). The groups were prepared as described above. The group listed as Insects 1 was those larvae grown for ⅔ of the five week growing period and the group listed as Insects 2 was those larvae that were grown for the full five week growing period. The Ergosterol content is shown as mg ergosterol/100 g dry matter (dm) in insect samples

TABLE 3

| | Ergosterol content mg/100 g DM |
|---|---|
| sample name | (average of three replicates ± SD) |
| Insects 1 | 32.96 ± 0.49 |
| Insects 2 | 37.57 ± 0.40 |
| Insect Control | 24.78 ± 0.11 |

As can be seen from the results shown in Table 3, the larvae that were fed fodder supplemented by the addition of mushroom biomass showed elevated levels of ergosterol relative to the control group indicating that the larvae were able to show elevated levels of ergosterol when they were fed fodder supplemented with the mushroom biomass.

Five grams of material was extracted with a methanol/dichloromethane 75:25 (v/v) mixture. The mixture was sonicated (40 kHz) for 10 min. After 2 hours, the extract was centrifuged at 12,000 rpm and then decanted from the supernatant. The extraction procedure was repeated twice, the resulting extracts were combined and evaporated to dryness.

Determinations were performed using high-performance liquid chromatography with a diode array detector (HPLC-DAD). The VWR/Hitachi LaChrom Elite liquid chromatography kit (Merck Hitachi, Tokyo, Japan) with DAD L-2455 detector was used for determinations. The column RP-18 4×250 mm (LiChrosfer) with grain size 5 μm, thermostat L-2350, pump L-2130, autosampler L-2200 were used.

The mobile phase comprised solvent A: methanol/water 80:20 (v/v) and solvent B: methanol/dichloromethane 75:25. The gradient program was: 0-10 min, 80: 20% B; 10-35 mins, 40-60% B; 35-50 minutes, 0-100% B; 50-55 minutes, 80-20% B; holding time of 15 min at 25° C.). The flow rate was 1.0 mL/min. Chromatographic peaks were recorded at 280 nm. The sterol standards were purchased from Fluka (Chemie AG).

Subsequently, tests were performed on the groups of the larvae testing for various vitamins to ascertain if those larvae that were fed fodder supplemented by mushroom biomass showed elevated levels of vitamins relative to those in a control group (i.e., the larval group that was fed fodder without the supplement from mushroom biomass).

Table 4 shows the content of selected vitamins in insect larvae fed with traditional fodder for the full rearing period (A) and fed with fodder with the addition of biomass after mushroom cultivation, depending on the stage of growth. Group 01 is those that were grown for ⅔ of the rearing period, and group 04 were those grown for the full rearing period. The vitamin amounts are shown in micrograms of vitamin per 100 g dry weight.

TABLE 4

| | | | | | Nicotinic | |
|---|---|---|---|---|---|---|
| | B1 | on average | B2 | on average | B3 | on average |
| A | 1094 | | 1986 | | 22915 | |
| | 1146 | | 2435 | | 21040 | |
| | 1010 | 1083 | 2143 | 2188 | 21802 | 21919 |
| 01 | 1895 | | 3365 | | 48102 | |
| | 1730 | | 2729 | | 50165 | |
| | 1760 | 1795 | 3011 | 3035 | 49597 | 49288 |
| 04 | 1935 | | 4205 | | 65303 | |
| | 1845 | | 4162 | | 70785 | |
| | 1897 | 1892 | 4129 | 4165 | 58279 | 64789 |

As should be apparent from Table 4, the addition of biomass from the addition of mushrooms to the fodder of the larval insects vastly and unexpectedly improved the presence of Thiamin (B1), Riboflavin (B2), and nicotinic acid relative to the control group.

### Thiamin (B1) and Riboflavin (B2)

Thiamine (B1) and riboflavin (B2) content was determined by HPLC. The HPLC samples were prepared in accordance with the methodology of PN-EN 14122: 2004/AC: 2006 and PN-EN 14152: 2004/AC: 2006. Thiamine and riboflavin were determined after the oxidation reaction before the column. For this purpose, a 0.04% solution of potassium hexacyanoferrate (III) in a 15% sodium hydroxide solution was added to the sample and it was shaken and left for 2 minutes. After adjusting the pH with orthophosphoric acid solution to 7, and centrifugation, the extracts were purified on SPE (solid phase extraction), centrifuged again and analyzed by HPLC. A Merck HITACHI liquid chromatograph (HPLC) equipped with a L-7612 on-line degasser, Programmable Autosampler L-7250, L-7100 pump, FL Detector L-7480 fluorescence detector,

Column Oven L-7360 Merck thermostat, Interface D was used for vitamin detection. The software detection program with software: D-7000 with HPLC-System-Manager (HSM) was used. The analysis was carried out on a Bionacom Velocity C18 PLMX 4.6×250 mm, 5 μm column from Bionacom LTD (Great Britain) together with a pre-column from the same company. The measurement was performed at the excitation and emission wavelengths:

360/503, enabling and allowing for the simultaneous determination of thiamine and riboflavin. The mobile phase was water with acetonitrile. Gradient elution was performed: t-0 w/ac 88/12; t=12 w/ac 0/100 at 22° C. Thiamine and riboflavin external standards in hydrochloric acid and acetic acid, respectively, were used to identify thiamine and riboflavin and to quantify them. Nicotinic acid (B3)

The content of vitamin B3 was determined by a modified method described by Juraj et al. (2003) using a Merck HITACHI liquid chromatograph (HPLC).

III. The aim of the next experiment was to demonstrate differences in the content of selected elements, organic compounds, vitamins and sterols in the body of larval forms of Alphitobius diaperinus species between individuals fed with feed mixtures using an additive in the form of biomass after cultivation and harvesting of A. bisporus mushrooms in the form of a substrate residue and stems of last flush mushrooms vs. only post-cultivation substrate (SMS) without any mushroom casing soil and stem residue.

The insects used in the experiments came from breeding. The experiments were carried out each time during the entire fattening period of the larval forms, i.e., from hatching from eggs to the larval forms ripening after a period of 5 weeks. The experiments were carried out in 8 repetitions, each time on 100 breeding containers for each group. They showed no differences in the values of BWG (Body Weight Gain)—body weight gain, FI (Food Intake)—amount of food consumed, FCR (Food Conversion Ratio) or length of fattening. The results were averaged.

The insects were bred in polypropylene containers with a bottom area of 0.24 m² at a temperature of 31° C. and 60% humidity throughout the fattening period, in the dark. The same starting amount of beetle eggs was used each time. After a 5-day incubation period, there was a 5-week fattening period. The feed was provided at 56-hour intervals depending on demand. The weight of the insects was determined after separating them from frass and food remains and weighing them at the end of the cycle. No dead insects were found in any of the samples. The following feed mixtures were used:

### Feed Mixture Composed of:

−35% by weight wheat bran, 60% by mass pulp from mushroom biomass after the last cut of cultivation (contains SMS residue and mushroom stems), 5% by weight feed supplement in the form of spent barley grains
B. Feed mixture composed of:
−35% by weight wheat bran, 60% by weight biomass pulp from clean SMS after cultivation (without any stems and casing soil), 5% by weight feed supplement in the form of barley spent grain brewer's

The pulp from the substrate was prepared as described—the raw substrate of the last flush was mechanically separated from the casing soil and stems of the mushrooms. Then, water was added to the substrate in a ratio of 1:1.5 by weight and the resulting mixture was ground to a uniform mass with granulation as described. The pulp was applied immediately after preparation at 56-hour intervals.

After the full fattening period, the larvae were separated from the frass and the remains of uneaten feed.

The larvae were dried and then ground. Samples were obtained from the material prepared in this way and analyzed using the methods previously described.

The content of selected ingredients were found in the meal of insects fed with feed mixture B. These results are shown in Tables C, D, and E.

TABLE C

| Compound (amino acid, amino acid | Relative Amount | |
|---|---|---|
| derivative, or steroid) | (DM = dry matter) | |
| ergothioneine | 7.57 +/− 0.38 | mg/100 g DM |
| phenylalanine | 380.88 ± 2.06 | mg/100 g DM |
| L-tryptophan | 144.34 ± 0.24 | mg/100 g DM |
| ergosterol | 15.90 ± 0.98 | mg/100 g DM |

TABLE D

| Element | Amount mg/kg dry weight) |
|---|---|
| Mg | 2457 +/− 9.4 |
| Zn | 42.5 +/− 0.1 |
| Cu | 17.6 +/− 1.1 |
| Fe | 37.0 +/− 0.6 |
| Ca | 1909 +/− 62.9 |
| At | 742.1 +/− 6.1 |
| K | 6529 +/− 157 |
| Mn | 3.4 +/− 04 |

TABLE E

| Vitamin | Relative Amount | |
|---|---|---|
| B1 | 1.89 | mg/100 g |
| B2 | 3.6 | mg/100 g |
| B3 | 56.9 | mg/100 g |

The analysis showed comparable vitamin content, lower ergosterol content, and much higher selected amino acids with significantly lower element content. Considering the dilution of the pulp which was present at a ratio of 1:1.5 is fully understandable, but it indicates also an extraordinary concentration of B vitamins and the tested amino acids in the mycelium itself.

IV. The aim of the next experiment was to ascertain if there were differences in the content of chitin in the bodies of Alphitobius diaperinus larvae between individuals fed with traditional feed mixtures and with the supplement of the biomass in the form of stems derived from A. bisporus after its harvesting process.

The insects used in the experiments came from the inventor's breeding inventory. The experiments were carried out each time throughout the fattening period of larval forms, i.e. from the time period starting with the hatching from eggs to the time period when the larval forms are mature (and are ready for processing) after a period of 5 weeks. The experiments were carried out in 8 repetitions, each time on 100 breeding containers for each group, and the respective groups showed no differences in the BWG (Body Weight Gain) values-weight gain, FI (Food Intake)—amount of food consumed, FCR (Food Conversion Ratio) or length of fattening. Accordingly, the differences seen in the relative amounts of the various biomolecules can be attributed to the different diets. The results have been averaged.

Insects were reared in polypropylene containers with a bottom surface area of 0.24 m² at a temperature of 30° C. and 60% humidity throughout the fattening period, in the dark. The same starting amounts of beetle eggs were used for each group. The 5-day incubation period was followed by a 5-week fattening period. The feed was supplied at 56 hour intervals depending on demand. The mass of insects was determined after separating them from their frass and the food remains and the larvae were weighed at the end of the cycle. No dead insects were found in any of the samples. As above, the following feed mixtures were used:

### Feed Mixture Composed of:

A. −35% by mass wheat bran, 60% by mass carrot pulp, and 5% by mass feed supplement in the form of barley brewer's spent grains.
B. Compound feed composed of:
−35% by mass wheat bran, 60% by mass pulp from mushroom biomass in the form of only stems after last cut of cultivation, and 5% by mass feed supplement in the form of barley brewer's spent grain.

The pulp from the stems of mushrooms was prepared as described, with the feed being applied immediately after preparation at 56-hour intervals. At the end of the fattening period, the larvae were separated from the frass and the rest of the unfinished feed. The larvae were dried and then ground. Samples were obtained from the material prepared in this way and then analyzed.

The chitin content test was performed according to a methodology enumerated by Research Institute of Feed Technology (Forschungsinstitut Futtermitteltechnik der IFF)

Frickenmühle 38110 Braunschweig-Thune Germany, as described in the below references, which are herein incorporated by reference in their entireties:

https://www.researchgate.net/publication/373317761_Chitin_in_Futtermitteln_-Analyse_Auswirkung_auf_den_Nahrwert_und_Kontrolle https://www.researchgate.net/publication/369977223_Bestimmung_des_Chitingehaltes_von_ins ektenbasierten_Produkten_Insekten_als_Proteintrager_Chitin_als_Nahrstoff https://www.researchgate.net/publication/363491517_Bestimmung_des_Chitingehaltes_von_ins ektenbasierten_Produkten

Briefly, the following procedure can be followed to determine the chitin content.

Grind the sample to a size that is less than about 1 mm.
Weigh 1.00 grams of sample into a fibrous bag.
Degrease (remove oils) in the sample using petroleum ether.
Perform alkaline hydrolysis using 0.25 M NaOH for 60 minutes at 100° C.
Remove the hydrolysis products by washing the resulting alkaline hydrolytic process twice with hot water.

The nitrogen content can be determined by a methodology enumerated by Kjeldahl. Briefly, three steps are performed: 1) digestion 2) distillation and 3) titration. The nitrogen species are converted to ammonia by wet combustion in the presence of a catalyst (such as selenium) and sulfuric acid and the ammonia is retained as ammonium sulfate. Treatment of the ammonium sulfate with

NaOH generates ammonia, which is distilled into boric acid. The borate anions formed are titrated with HCL, which allows the determination of nitrogen content.

The chitin content can be determined by using the following formula.

$$Chitin ( \% ) = ( ( V ‐ Vblind ( ml ) * 100 * 1.4007 / m sample ( mg ) * 14.007 ( g / mol N ) ) ( 203.19 ( g / mol GlcNAc ) )$$

The results unexpectedly showed not only a significant increase in the chitin content in insects fed with mixture B, but also an increase in protein content with a reduction of fat content (see table 5):

TABLE 5
All samples were analyzed for their basic protein
and fat components according to the LUFA methods.

| | | Values | | | | | | | |
|---|---|---|---|---|---|---|---|---|---|
| Analysis | sample | 1 | 2 | 3 | 4 | x̅ | s | n | Units |
| L/H₂O-Tr | A | 4.9 | 4.9 | | | 4.9 | | 2 | % |
| moisture | | | | | | | | | |
| L/CH-RP- | | 57.1 | 57.6 | | | 57.4 | | 2 | % |
| TE | | | | | | | | | |
| raw protein | | | | | | | | | |
| L/CH-Ft | | 25.2 | 25.33 | | | 25.3 | | 2 | % |
| raw fat | | | | | | | | | |
| L/CH-Chitin | | 7.8 | 7.9 | | | 7.9 | | 2 | % |
| L/H₂O-Tr | B | 5.4 | 5.4 | | | 5.4 | | 2 | % |
| moisture | | | | | | | | | |
| L/CH-RP- | | 60.8 | 60.7 | | | 60.8 | | 2 | % |
| TE | | | | | | | | | |
| raw protein | | | | | | | | | |
| L/CH-Ft | | 19.73 | 19.87 | | | 19.8 | | 2 | % |
| raw fat | | | | | | | | | |
| L/CH-Chitin | | 9.4 | 9.3 | | | 9.4 | | 2 | % |

V. In a further example of performance, an experiment was conducted with the aim of demonstrating an increase in melanins content in larval forms of Hermetia illucens fed a feed mixture with mushroom biomass additive that was attained after cultivation and processing of A. bisporus mushrooms.

The reason for the tests was the marked change in coloration of larvae various species fed with mushroom biomass compared to larvae fed with mixtures containing no mushrooms. Depending on the growth stage, the larvae BSF have a light brown color, gradually turning dark brown at the pre-pupae stage. From the inventor's observations, it was apparent that larvae fed on feed mixtures with added fungal biomass immediately gain a dark brown color, turning almost black at the pre-pupae stage. Individuals at this developmental stage more closely resembled adults-imago.

The experiments were carried out throughout the rearing period of the larval forms, i.e., at time periods after the hatching of eggs. The larvae were allowed to grow using the feed disclosed herein for a period of 14 days (including 3 days incubation period).

Insects were reared in polypropylene containers with a bottom surface area of 0.64 m² at a temperature of 32-34° C. and 75-85% humidity throughout the fattening period. The same starting amounts of eggs were used each time. The quickly spoiled feed supplement contains mushroom stems and slaughterhouse waste in the form of pulp was fed at 48 hour intervals depending on demand.

The experiments were carried out using 3 repetitions, each time using 10 breeding containers for each group.

The insects were separated from frass and the food remains and weighed at the end of the cycle. No dead insects were found in any of the samples. The following feed mixtures were used:

### A. Feed Mixture Consisting of:

−75% crushed and hydrated wheat grains and wheat bran, 20% by weight of feed additive in the form of barley brewer's spent grains, 5% pulp from slaughterhouse waste, especially guts, heads, feet, and/or poultry bodies
B. Feed mixture consisting of:
−75% shredded and hydrated mushroom biomass after the last crop swath (includes SMS and mushroom stems), 20% by weight of feed additive in the form of barley brewer's spent grains, 5% pulp from slaughterhouse waste, especially guts, heads, feet, and/or poultry bodies.

At the end of the fattening period, the larvae before pre-pupae stage were separated from the frass and the rest of the unfinished feed. The larvae were dried.

The test material was micronized/crushed to a particle size=3-4 mm. Then 10% NaOH was added to this powder in a ratio of 1:10 of the mass to be deproteinized, in order to isolate chitin with repeated stirring at 80° C. in a closed vessel without air for 1.5 hours. Then, after cooling, the same amount of 50% NaOH was added to the same vessel and chitin was deacylated in a closed vessel without air at 95° C. for 2.0 hours with repeated stirring. The resulting hydrolysate was filtered in a Buchner funnel. The residue (chitosan) was washed at pH=7 until the water with which this sample was washed had a neutral pH. Melanins were removed from the alkaline hydrolysate by sedimentation with concentrated hydrochloric acid at pH 2, and then centrifuged at 1500 g for 15 minutes. The isolated residue was washed until a neutral pH was attained. The residue was dried at 50° C. to a constant weight (until there was no more loss of mass) or added to previously isolated chitosan to obtain a melanin-chitosan complex, which was also dried at 50° C. When the melanin residue was centrifuged, it was separated into two fractions—the actual residue and the hydrophobic fraction over the supernatant (i.e., the melanin layer and the fat complex layer). After the residue was separated, a separation funnel was used to isolate the melanin and fat complex. The isolated fractions were dried as described above. Identification of the pigments was based on evaluation of their solubility, and their spectral and paramagnetic properties. Electron paramagnetic resonance (EPR) spectra were then measured for the dry samples. The obtained spectra were compared with the standard to identify the various components.

Extraction of lipids from the melanin-fat complex was carried out in a glass ampoule with a screw cap by adding 3 ml of DCM-methanol (dichloromethane-methanol) mixture (volume 10:1) and treated in an ultrasonic bath for 15 minutes. The specific parameters of the gas chromatograph and the parameters for recording the mass spectra were then described.

The melanin-like pigment was isolated from both samples of H. illucens larvae. The EPR signal of the pigment of sample A larvae was, with a g-factor of 2.0036 and ΔH value of 6.3±0.7 Gs. It was assumed that the pigment from the larvae of sample A, belongs to the class of eumelanins. The concentration of paramagnetic centers in the sample was 1.1×10¹⁶ spin/g dry weight, corresponding to 5.7 mg of eumelanin per 1 g of larvae dry weight.

Melanin from the sample of insects fed with feed mix B was: AH-5.7 Gs, g-factor-2.0036, spin concentration is 8,0 ×10¹⁶ spin/g per dry weight of the sample, which corresponds to an eumelanin content of 22 mg eumelanin/g DM.

The nearly 4-fold increase in eumelanin content among larvae fed with a mixture using biomass containing the stems and mycelium of the double-spore mushroom justifies the use of this method of obtaining it on an industrial scale.

In almost all instances, the presence of mushroom biomass supplementing the fodder for the various larval insect groups showed elevated levels of elements, amino acids, vitamins and/or steroids relative to the control groups that were fed fodder that did not contain the mushroom biomass. The insects were able to process and store the additional elements and biomolecules so that they were present in the growing larvae.

Thus, in an embodiment, the present invention relates to tests conducted on the feeding of various species of insects used in industrial production with a by-product of mushroom cultivation in the form of a substrate after cultivation and processing waste of mushrooms (Agaricus bisporus).

In an embodiment, the present invention shows unexpectedly superior results and shows that desired substances can be recovered that have both dietary and medicinal properties from the biomass of insects after their cultivation. The processing of mushrooms to effectuate a biological transfer of these desired substances to the body of insects can not only be attained, but their intensification can be realized.

In an embodiment, the present invention shows that it is possible to obtain insect meat with previously unknown quality parameters by supplementing their feed with mushroom biomass, far exceeding any product obtained in the traditional fattening process of insect larvae with the use of commonly used feed ingredients.

In an embodiment, the present invention relates to insect larvae that have been fed fodder comprising mushroom biomass, wherein said insect larvae comprise elevated levels of one or more of elements, amino acids, vitamins, and/or sterols relative to insect larvae that have been fed fodder that is free of mushroom biomass.

In an embodiment, the mushrooms that can be used in connection with the present invention are edible mushrooms. Edible mushrooms that can be used in the present invention include but are not limited button mushrooms (Agaricus bisporus), Cremini (Italian Brown) Mushrooms, Portobello Mushrooms, Shiitake Mushrooms (Forest or Oak), Oyster Mushrooms, Porcini Mushrooms, Morel Mushrooms, Enoki (Snow Puff) Mushrooms, Chanterelle (Girolle) Mushrooms, and/or Maitake Mushrooms.

In a variation, the insect larvae are Coleoptera, Diptera, Orthoptera insects. In a variation, the Coleoptera, Diptera and Orthoptera insects are Alphitobius diaperinus, Alphitobius laevigatus, Tenebrio molitor, Tenebrio obscurus, Tenebrio opacus, Zophobas atratus, Zophobas morio, Hermetia illucens, Musca domestica, Acheta domestica, Gryllodes sigillatus, Gryllus assimilis, Gryllus bimaculatus, Locusta migratoria, Schistocerca gregaria.

In a variation, the mushroom biomass is derived from Agaricus bisporus. In an embodiment, the mushroom biomass is derived from the mushroom, the mycelium, stems. In a variation, the elements are one or more of potassium, calcium, manganese, iron, copper, zinc or rubidium. In a variation, the amino acids are essential amino acids. In a variation, the amino acids are tryptophan, phenylalanine, or ergothioneine. In a variation, the vitamins are B1, B2, or nicotinic acid. In a variation, the sterol is ergosterol.

In an embodiment, the present invention relates to a method of increasing a level of elements, amino acids, vitamins, and/or sterols in insect larvae relative to a control group of insect larvae, the method comprising:
procuring the eggs of insects,
allowing the eggs to hatch to insect larvae, and
growing the insect larvae in the presence of fodder that comprises mushroom biomass.

In a variation of the method, the insect larvae are Coleoptera and/or Diptera and/or Orthoptera insects. In a variation, the mushroom biomass is derived from Agaricus bisporus. In a variation of the method, the mushroom biomass is derived from the mushroom, the mycelium, and stems. In a variation, the elements are one or more of potassium, calcium, manganese, iron, copper, zinc or rubidium. In a variation of the method, the amino acids are essential amino acids. In a variation, the amino acids are tryptophan, phenylalanine, or ergothioneine. In a variation of the method, the vitamins are B1, B2, or nicotinic acid. In a variation, the sterol is ergosterol.

In an embodiment, the present invention relates to a method of increasing the level of elements, amino acids, vitamins, and/or sterols in a human, said method comprising ingesting insect larvae that have elevated levels of the elements, amino acids, vitamins, and/or sterols, wherein said insect larvae are Coleoptera and/or Diptera and/or Orthoptera insects that have been fed fodder that comprises mushroom biomass derived from Agaricus bisporus. In a variation of the method, the elements are one or more of potassium, calcium, manganese, iron, copper, zinc or rubidium, the amino acids are essential amino acids, the vitamins are one or more of B1, B2, or nicotinic acid, and the sterol is ergosterol. In a variation, the amino acids are one or more of tryptophan, phenylalanine, or ergothioneine.

In an embodiment, the present invention relates to a method of transferring elevated levels of the elements, amino acids, vitamins, and/or sterols from a mushroom to insect larvae, the method comprising feeding the insect larvae with fodder that comprises mushroom biomass. In a variation of the method, the insect larvae are Coleoptera and/or Diptera and/or Orthoptera insects that have been fed fodder that comprises mushroom biomass derived from Agaricus bisporus. In a variation of the method, the elements are one or more of potassium, calcium, manganese, iron, copper, zinc or rubidium, the amino acids are essential amino acids, the vitamins are one or more of B1, B2, or nicotinic acid, and the sterol is ergosterol. In a variation, the amino acids are one or more of tryptophan, phenylalanine, and/or ergothioneine.

The below enumerated species are all mushrooms that are of industrial importance, cultivated for food and medical purposes, whose mycelium, stems, processing waste and substrates parts after cultivation can be used to transfer ingredients with dietary and medicinal properties into an insect bodies.

In an embodiment, the present invention relates to a method of increasing levels of chitin and protein to insect larvae, the method comprising feeding the insect larvae with fodder that comprises mushroom biomass.

In an embodiment, the mushrooms that can be used in the present inventio include reishi, shitake, and/or maitake mushrooms. In an embodiment, the mushrooms that can be used include Pleurotus ostreatus (Jacq.) P. Kumm.); Pleurotus citrinopileatus (Singer); Pleurotus florida (Singer); Pleurotus pulmonarius (Fr.) Quél.; Pleurotus eryngii (DC.) Quél.; Pleurotus djamor (Rumph. Ex Fr.) Boedijn; Hypsizygus ulmarius (Bull.) Redhead; Coprinus comatus (O.F. Müll.) Pers.; Ganoderma lucidum (Curtis) P. Karst; Cordyceps militaris (L.) Link; Cordyceps sinensis; Agaricus subrufescens Peck; Stropharia rugosoannulata Farl. ex Murrill; Flammulina velutipes (Curtis) Singer; Hypsizygus tessellatus (Buli.) Singer and Hypsizygus marmoreus (Buli.) Singer (Buna shimeji); Cyclocybe aegerita (cylindracea) (V. Brig.) Vizzini; Sparassis crispa (Wulfen) Fr.; Hericium coralloides (Scop.) Pers.; Hericium erinaceus (Buli.) Persoon; Tuber borchii Vittad.; Tuber indicum; Polyporus umbellatus (Pers.) Fr.; Grifola frondose (Dicks.) Gray; Laetiporus sulphureus (Bull.) Murrill.; Ganoderma lucidum (Curtis) P. Karst; Pholiota nameko (T. Itô) S. Ito et S. Imai.; Kuehneromyces mutabilis (Schaeff.) Singer et A. H. Sm.; Volvariella volvacea (Bull.) Singer; Hericium coralloides (Scop.) Pers.; Hericium erinaceus (Buli.) Persoon; Lentinula edodes (Berk.) Pegler (Shiitake); Auricularia polytricha (Mont.) Sacc (mun mushroom); Grifola frondosa (Dicks.) Gray, Psilocybe cubensis; Psilocybe semilanceata; Psilocybe mexicana, Psilocybe muscorumi; Stropharia cubensis or Fusarium sp.

In one embodiment, the biomolecules that are of interest for transfer from the enumerated mushrooms to the insects are:
5-hydroxytryptophan, L-tryptophan, serotonin, melatonin, ecdysone, ergosterol, lovastatin, ergothioneine, GABA, phenolic compounds, melanin, cordycepin, erinacin, hericenon, mescaline, enzymes, B vitamins, and macro- and microelements.

In an embodiment, the present invention relates to a feed for feeding insect larvae, said feed comprising fodder and mushroom biomass. In a variation, the feed comprises wheat bran, pulp from mushroom biomass after cultivation and mushroom stems, and a feed supplement. In a variation, the feed comprises about 35% by weight wheat bran, 60% by weight pulp from mushroom biomass after cultivation and mushroom stems, and 5% by mass feed supplement, wherein the feed supplement may be brewer's barley spent grain and/or a meat sidestream, that can be used as a protein supplement.

In an embodiment, the feed may contain from 20-70% by weight wheat bran, 20-75% mushroom biomass, and 1 to 25% by weight a mass feed supplement such as barley spent grain. In a variation, the feed may contain from 30-40% by weight wheat bran, 50-70% mushroom biomass, and 5 to 25% by weight a protein mass feed supplement such as barley spent grain and/or a meat sidestream.

In an embodiment, the present invention relates to a feed mixture that optionally comprises brewer's spent grain, and comprises 1-25% of feed yeast and/or yeast mass. The yeast mass can be derived as a yeast jelly like, semi liquid mass on the bottom of a beer brewing tank after brewing. This feed mixture can be used at both the stage of rearing the larval forms and/or at the reproduction imago stage.

The following reference(s) and any reference referred to herein is incorporated by reference in its entirety for all purposes.

Juraja S M, Trenerry V C, Millar R G, Sheelings P, Buick D R. Asia Pacific food analysis network (APFAN) training exercise: the determination of niacin in cereals by alkaline extraction and high performance liquid chromatography. J Food Compos Anal. (2003), 16, 93-106. doi: 10.1016/S0889-1575 (02) 00131-X.

It should be understood, and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

We claim:

1. Enriched insects that have been fed fodder comprising mushroom biomass, wherein said enriched insects comprise elevated levels of one or more protein, chitin, elements, amino acids, vitamins, sterols and/or chemical compounds relative to insects of a same species and at the same developmental stage that have been fed fodder that is free of mushroom biomass.

2. The enriched insects of claim 1, wherein the enriched insects are from Coleoptera, Diptera and/or Orthoptera orders.

3. The enriched insects of claim 1, wherein the mushroom biomass is derived from Agaricus bisporus, Agaricus blazei, Pleurotus ostreatus, Pleurotus citrinopileatus, Pleurotus florida, Pleurotus pulmonarius, Pleurotus eryngii, Pleurotus djamor, Agrocybe aegerita, Hypsizygus ulmarius, Coprinus comatus, Ganoderma lucidum, Cordyceps militaris, Cordyceps sinensis, Agaricus subrufescens, Stropharia rugosoannulata, Flammulina velutipes, Hypsizygus tessellatus, Hypsizygus marmoreus, Cyclocybe aegerita (cylindracea), Sparassis crispa, Hericium coralloides, Hericium erinaceus, Tuber borchii, Tuber indicum, Polyporus umbellatus, Grifola frondose, Laetiporus sulphureus, Ganoderma lucidum, Pholiota nameko, Kuehneromyces mutabilis, Volvariella volvacea, Hericium coralloides, Hericium erinaceus, Hericium abietis, Lentinula edodes, Auricularia polytricha, Grifola frondose, Psilocybe cyanescens, Psilocybe cubensis, Psilocybe semilanceata, Psilocybe mexicana, Psilocybe muscorumi, Stropharia cubensis, Trametes versicolor, Innonotus obliquus, Coriolus versicolor, Flammulina velutipes, or Phellinus linteus.

4. The enriched insects of claim 1, wherein the mushroom biomass comprises mushroom fruitbody and/or mycelium and/or stems.

5. The enriched insects of claim 3, wherein the insects are insect larvae and/or imago, wherein the mushroom biomass is derived from Agaricus bisporus.

6. The enriched insects of claim 2, wherein the enriched insects are one or more species selected from the group consisting of Alphitobius diaperinus, Alphitobius laevigatus, Tenebrio molitor, Tenebrio obscurus, Tenebrio opacus, Zophobas atratus, Zophobas morio, Hermetia illucens, Musca domestica, Acheta domestica, Gryllodes sigillatus, Gryllus assimilis, Gryllus bimaculatus.

7. The enriched insects of claim 1, wherein the elements comprise one or more of potassium, calcium, manganese, iron, copper, zinc or rubidium, the amino acids comprise essential amino acids, the vitamins comprise B1, B2, nicotinic acid and/or niacin, and the organic chemical compounds comprise melanin or chitin.

8. The enriched insects of claim 1, wherein the one or more elements, amino acids, vitamins, chemical compounds and/or sterols reach a peak maximal amount during a fattening period relative to an amount at an end of the fattening period.

9. The enriched insects of claim 1, wherein the one or more elements, amino acids, vitamins, chemical compounds and/or sterols are one or more members selected from the group consisting of 5-hydroxytryptophan, L-tryptophan, serotonin, melatonin, ecdysone, ergosterol, lovastatin, ergothioneine, GABA, phenolic compounds, cordycepin, eranacines, hericenones, psylocibin, psilocin, enzymes, B vitamins, macro-elements, microelements, chitin, beta-glucan, triterpenes, melanin, tryptophan, phenylalanine, ergothioneine, and ergosterol.

10. The enriched insects of claim 1, wherein the protein is increased.

11. A method of increasing the level of elements, amino acids, vitamins, and/or sterols in insect larvae and/or in other animals or humans, said method comprising:
procuring the eggs of insects,
allowing the eggs to hatch to insect larvae,
growing the insect in the presence of fodder that comprises mushroom biomass to generate enriched insects, and optionally, having the humans directly or indirectly ingest the enriched insects.

12. The method of claim 10, wherein the insect larvae are Coleoptera, Orthoptera or Diptera insects.

13. The method of claim 10, wherein the mushroom biomass is derived from Agaricus bisporus.

14. The method of claim 10, wherein the elements comprise one or more of potassium, calcium, manganese, iron, copper, zinc or rubidium, the amino acids comprise one or more essential amino acids, the vitamins comprise B1, B2, or nicotinic acid, and the sterol comprises egosterol.

15. The method of claim 10, wherein the amino acids comprise one or more of tryptophan, phenylalanine, or ergothioneine.

16. The method of claim 10, wherein said insects are Coleoptera and/or Diptera and/or Orthoptera insects that have been fed fodder that comprises the mushroom biomass derived from Agaricus bisporus.

17. The method of claim 15, wherein the elements are one or more of potassium, calcium, manganese, iron, copper, zinc or rubidium, the amino acids are essential amino acids, the vitamins are one or more of B1, B2, or nicotinic acid, and the sterol is ergosterol.

18. A feed for feeding insects, said feed comprising bran and/or any other cereals and their milling products, pulp from mushroom biomass after cultivation (spent mushroom substrate) and/or mushroom stems, and a feed supplement.

19. The feed of claim 18, wherein the feed comprises about 30-35% by weight wheat bran, 55-60% by weight pulp from mushroom biomass after cultivation and/or mushroom stems, and 5-15% by mass feed supplement, wherein the feed supplement comprises brewer's barley spent grain and/or dry feed yeast and/or wet yeast mass, and/or animal protein in the form of pulp derived from slaughterhouse waste, fish waste, and/or former meat food.

20. The feed of claim 18, wherein the feed comprises about 30-50% by weight wheat bran and/or any other cereals and their milling products and/or brewer's barley spent grain, and further comprises 50-70% by weight leachate/filtration from mycoprotein crops and/or water after rinsing mushrooms and/or water used to blanch mushrooms, and/or fluids from mycelial in in vitro crops.
